(19) United States
(12) Patent Application Publication
(10) Pub. No.: US 2014/0220684 A1
(43) Pub. Date: Aug. 7, 2014

(54) COMPOSITION COMPRISING OXYGEN-RELEASING MICROCAPSULES CONTAINING HYDROGEN PEROXIDE FOR ENHANCING CELL PROTECTION AND VIABILITY AT ROOM TEMPERATURE

(71) Applicants: KYUNGPOOK NATIONAL UNIVERSITY HOSPITAL, Daegu (KR); KYUNGPOOK NATIONAL UNIVERSITY INDUSTRY-ACADEMIC COOPERATION FOUNDATION, Daegu (KR)

(72) Inventors: Jeong Ok LIM, Daegu-si (KR); Chong Lau HUI, Daegu (KR)

(21) Appl. No.: 14/067,658

(22) Filed: Oct. 30, 2013

(30) Foreign Application Priority Data
Feb. 6, 2013 (KR) 10-2013-0013468

(51) Int. Cl. C12N 5/071

(52) U.S. Cl. CPC C12N 5/0602
USPC 435/374

(57) ABSTRACT

The present invention provides a composition comprising oxygen-releasing microcapsules containing hydrogen peroxide for protection of cells at room temperature. The oxygen-releasing microcapsules containing hydrogen peroxide according to the present invention has an excellent effect of improving the viability of cells at room temperature and thus can be effectively used as a composition for protection of cells in manipulating, storing, or transporting cells at room temperature.

### CROSS-REFERENCE TO RELATED PATENT APPLICATION

This application claims the benefit of Korean Patent Application No. 10-2013-0013468, filed on Feb. 6, 2013, in the Korean Intellectual Property Office, the disclosure of which is incorporated herein in its entirety by reference.

### BACKGROUND OF THE INVENTION

1. Field of the Invention

The present invention relates to a composition comprising oxygen-releasing microcapsules containing hydrogen peroxide for protection of cells at room temperature.

2. Description of the Related Art

Recently, research using cells has continued to progress in the biotechnology industry. In particular, the research and industrialization of cell therapy using stem cells has been actively conducted. During the stem cell research, when cells or tissues are harvested from the body and left at room temperature without supplying oxygen for a long time, the cells or tissues would undergo apoptosis due to hypoxia, and it is very difficult to transport cells or tissues at room temperature without freezing. Moreover, during the process of freezing and thawing cells or tissues, the viability of cells or tissues is reduced, and thus it is very important to solve this problem.

Accordingly, the present inventor has made efforts to solve the above-described problem and develop a composition for protection of cells at room temperature and found that the viability of cells at room temperature is improved when oxygen-releasing microcapsules containing hydrogen peroxide was prepared and the cells were treated with the microcapsules, thus completing the present invention.

### SUMMARY OF THE INVENTION

An object of the present invention is to provide a composition comprising oxygen-releasing microcapsules containing hydrogen peroxide for protection of cells at room temperature.

### BRIEF DESCRIPTION OF THE DRAWINGS

The above and other features and advantages of the present invention will become more apparent by describing in detail exemplary embodiments thereof with reference to the attached drawings in which:

FIG. 1 is a conceptual view of oxygen-releasing microcapsules containing hydrogen peroxide of the present invention;

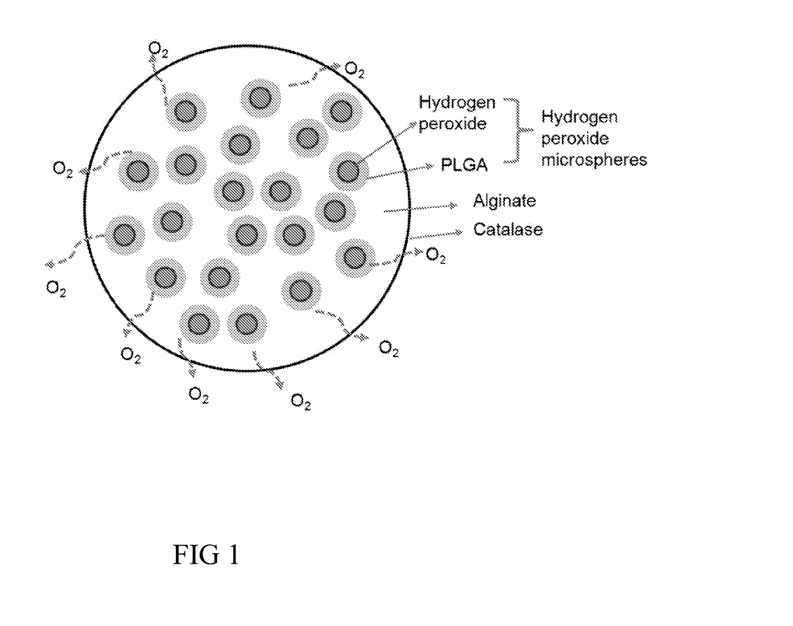

FIG. 2 is a view showing the morphology of amniotic fluid-derived stem cells treated with microcapsules containing hydrogen peroxide (10 mg) and cultured at room temperature for 24 hours and 48 hours;

FIG. 3 is a view showing the results of CCK assay showing the viability of amniotic fluid-derived stem cells in different concentrations (1×10³ cells, 1×10⁴ cells, and 1×10⁵ cells) treated with different amounts (5 mg, 10 mg, and 15 mg) of microcapsules containing hydrogen peroxide and cultured at room temperature for 24 hours and 48 hours; and

FIG. 4 is a view showing the release of oxygen from microcapsules containing hydrogen peroxide over time.

### DETAILED DESCRIPTION OF THE INVENTION

Hereinafter, the present invention will be described in detail with reference to the accompanying drawings.

The present invention provides a composition comprising oxygen-releasing microcapsules containing hydrogen peroxide for protection of cells at room temperature.

In the present invention, the term “room temperature” means the ambient temperature of a room, preferably 15 to 25° C.

Cells used in the present invention include, but are not limited to, all adult cells, such as muscle cells, adipocyte, skin cells, nerve cells, liver cells, etc., and stem cells. The term “stem cells” refers to cells having the ability of self-replication and the ability of differentiation into at least two cells, and examples of stem cells include, but are not limited to, umbilical cord-derived stem cells, cord blood-derived stem cells, bone marrow-derived stem cells, adipose-derived stem cells, muscle-derived stem cells, neural-derived stem cells, skin-derived stem cells, amniotic fluid-derived stem cells, and placenta-derived stem cells.

In the present invention, it is preferred that the content of hydrogen peroxide is 5 to 15 wt %, preferably 10 wt %, with respect to the total weight of the microcapsules when the cell number is 10⁵ to 10⁹. If the content of hydrogen peroxide is less than 5 wt %, the cell protective effect is insignificant, whereas if the content of hydrogen peroxide exceeds 15 wt %, it may cause oxidative stress to cells. When the number of cells varies, the amount of hydrogen peroxide should be increased or decreased depending on the variation.

The oxygen-releasing microcapsules containing hydrogen peroxide according to the present invention have an excellent effect of improving the viability of cells at room temperature and thus can be effectively used as a composition for protection of cells in manipulating, storing, or transporting cells at room temperature.

The following examples are provided to aid the understanding of the present invention. However, the following examples are only for the understanding of the present invention, and the present invention is not limited by the following examples.

### EXAMPLE 1

### Preparation of Microcapsules Containing Hydrogen Peroxide

In order to prepare microcapsules containing hydrogen peroxide, the following experiment was performed. First, 6 ml of 4% hydrogen peroxide (Aldrich, USA) was emulsified with 1.5 ml of poly lactic-co-glycolic acid (PLGA, LA:GA, 50:50, MW=11,000 gmol⁻¹, Boehringer Ingelheim, Germany) dissolved in dichloromethane (DCM) and followed by 4 ml of 3.5% polyvinyl alcohol (PVA, Sigma, USA). The mixture was stirred at room temperature for 4 hours to allow evaporation of the organic solvent, thus preparing PLGA microspheres containing hydrogen peroxide. The PLGA microspheres containing hydrogen peroxide were mixed with 1% alginate on which 3% catalase (Aldrich, USA) was immobilized. The resulting mixed solution was cross-linked with 1% calcium chloride (Aldrich, USA) using a dripping method to be encapsulated. Here, the catalase was used as an enzyme that specifically decomposes hydrogen peroxide into oxygen, and the encapsulated microspheres were rinsed with water and PBS, thus preparing the microcapsules containing hydrogen peroxide.

### EXPERIMENTAL EXAMPLE 1

Determination of Effect of Microcapsules Containing Hydrogen Peroxide on Cell Viability at room temperature

In order to determine the effect of the microcapsules containing hydrogen peroxide obtained in Example 1 on the viability of amniotic fluid-derived stem cells at room temperature, the microcapsules containing 30 mg of hydrogen peroxide were treated on 10⁵/ml cells and cultured at room temperature for 24 hours and 48 hours, and then the state of amniotic fluid-derived stem cells was observed under a microscope. As a result, no significant difference was observed compared to the group without the microcapsules containing hydrogen peroxide, and apoptosis was observed in both groups. Moreover, after 48 hours of the cultivation, morphological changes in amniotic fluid-derived stem cells were observed, and shrinkage of the cells was observed.

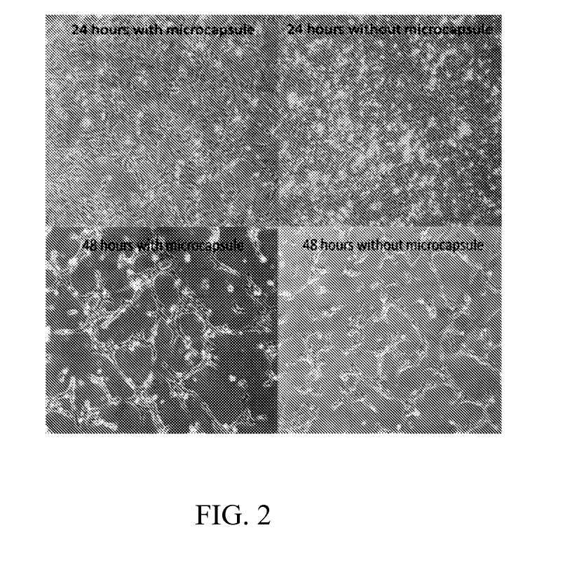

Based on the above experimental results, cholecystokinin (CCK) assay (Dojindo, USA) was performed on 10⁵/ml cells treated with the microcapsules containing 10 mg of hydrogen peroxide by varying the amount of hydrogen peroxide contained in the medium. Then, the state of amniotic fluid-derived stem cells was observed under a microscope, and the results are shown in FIG. 2.

As shown in FIG. 2, upon treatment with 10 mg of hydrogen peroxide, the attachment and viability of amniotic fluid-derived stem cells were increased compared to the group without the microcapsules containing hydrogen peroxide.

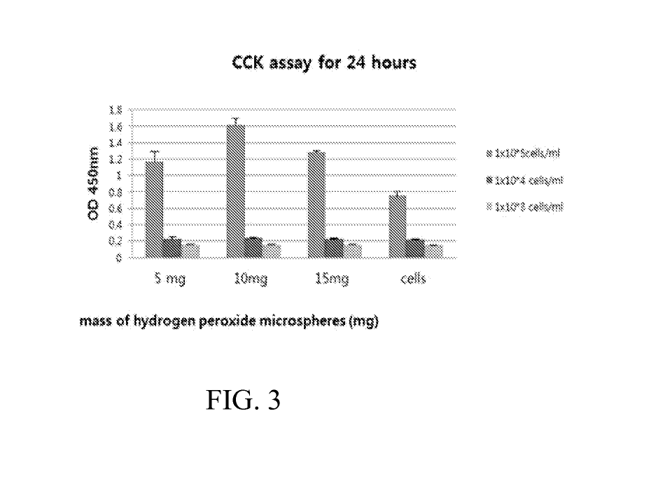

Moreover, the CCK assay was performed by varying the mass of microcapsules containing hydrogen peroxide to 5 mg, 10 mg, and 15 mg, and the results are shown in FIG. 3.

As shown in FIG. 3, it was found that the increase in the mass of microcapsules containing hydrogen peroxide contributed to the increase in cell viability. In particular, upon treatment with the microcapsules containing 10 mg of hydrogen peroxide, the cell viability was 50% higher than that of the control group.

### EXPERIMENTAL EXAMPLE 2

Determination of Release of Oxygen from Microcapsules Containing Hydrogen Peroxide

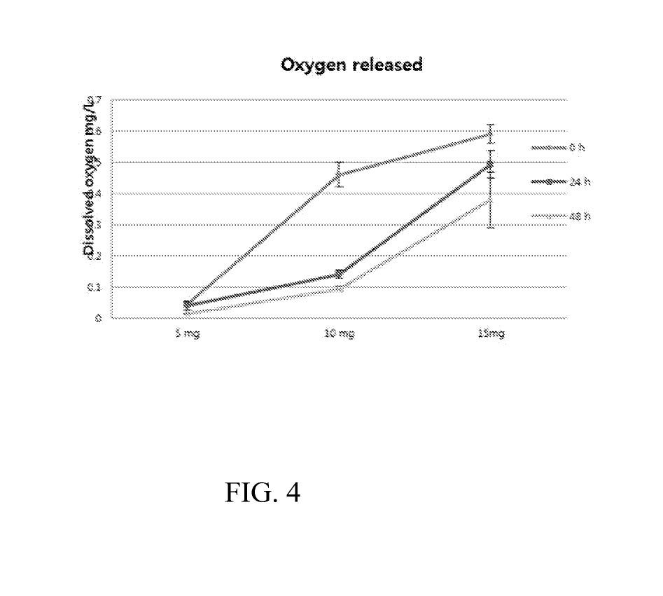

In order to determine the release of oxygen from the microcapsules containing hydrogen peroxide obtained in Example 1, the amount of oxygen released from the microcapsules containing hydrogen peroxide depending on the mass of microcapsules containing hydrogen peroxide was measured using a portable oxygen meter (Thermo, ORION series, 3 Star), and the results are shown in FIG. 4.

As shown in FIG. 4, it was found that the release of oxygen was correlated to the content of hydrogen peroxide and the release of oxygen lasted for 48 hours when the content of hydrogen peroxide was more than 10 mg. In particular, the microcapsules containing 15 ml of hydrogen peroxide showed much consistent release of oxygen.

Based on the above experimental results, it was found that the microcapsules containing hydrogen peroxide according to the present invention has the oxygen release function and has an excellent effect of improving the viability of cells at room temperature by supplying an appropriate amount of oxygen that does not cause oxidative stress to cells. Therefore, the microcapsules containing hydrogen peroxide according to the present invention can be used in manipulating or transporting cells or tissues at room temperature without freezing and can be used to improve the viability of cells or tissues that undergo hypoxia.

As described above, the microcapsules containing hydrogen peroxide according to the present invention has an excellent effect of improving the viability of cells at room temperature and thus can be effectively used as a composition for protection of cells in manipulating, storing, or transporting cells at room temperature.

What is claimed is:

1. A composition comprising oxygen-releasing microcapsules containing hydrogen peroxide for protection of cells at room temperature.

2. The composition of claim 1, wherein the room temperature is 15 to 25° C.

3. The composition of claim 1, wherein the cells comprise at least one selected from the group consisting of muscle cells, adipocyte, skin cells, nerve cells, liver cells, and stem cells.

4. The composition of claim 3, wherein the stem cells comprise at least one selected from the group consisting of umbilical cord-derived stem cells, cord blood-derived stem cells, bone marrow-derived stem cells, adipose-derived stem cells, muscle-derived stem cells, neural-derived stem cells, skin-derived stem cells, amniotic fluid-derived stem cells, and placenta-derived stem cells.

5. The composition of claim 1, wherein the hydrogen peroxide is contained in an amount of 5 to 15 wt % with respect to the total weight of the microcapsules.

6. The composition of claim 1, wherein the composition is used in storing or moving cells at room temperature.
